(19) United States
(12) Patent Application Publication
(10) Pub. No.: US 2024/0398609 A1
(43) Pub. Date: Dec. 5, 2024

(54) BODY-WORN URINAL FOR FEMALE PATIENT

(71) Applicants: MEDIRUNS CO., LTD., Bucheon-si (KR); MEDIRUNS CO., LTD., Gyeonggi-do (KR)

(72) Inventor: Bong Joo LEE, Bucheon-si (KR)

(21) Appl. No.: 18/700,353

(22) PCT Filed: Dec. 9, 2021
(86) PCT No.: PCT/KR2021/018568

§ 371 Date: Apr. 11, 2024

(30) Foreign Application Priority Data
Dec. 7, 2021 (KR) 10-2021-0174114

(51) Int. Cl. A61F 5/455

(52) U.S. Cl. CPC A61F 5/455

(57) ABSTRACT

The present invention relates to a body-worn urinal for a female patient, wherein same can be easily worn not only when the patient is lying, but also moving; same does not cause irritation, pain, or other types of inconvenience; even when worn for a long period of time, same has excellent ventilation; an embedded infrared sterilizer can be used to sterilize same and for treatment; and same thus enables both natural urination and sanitary maintenance/management. The present invention provides a body-worn urinal for a female patient, comprising: a urination cup for tightly guiding female urination; a connecting adapter installed to be fastened to the front of the urination cup; a urine collecting tube, one end of which is fastened to the connecting adapter, and the other end of which has a spherical inner space, the urine collecting tube being installed such that urine is discharged through a drainage part inclined downwards in the space; a urine discharge tube connected/installed to discharge urine collected in the urine collecting tube; and a urine container connected to the urine discharge tube to temporarily store urine. The body-worn urinal for a female patient further comprises: a far-infrared sterilizer mounted through one end of the upper side of the urine collecting tube and installed for sterilization and treatment; and a diagnostic test kit inserted/coupled to one side of the urine container for diagnostic test of urine.

### TECHNICAL FIELD

The present invention relates to a body-worn urinal for female patients, and more specifically, to a body-worn urinal for female patients, which is easy to wear even when the patient is lying down or moving, and which is capable of ventilation even when being worn for a long time without causing discomfort due to a foreign body sensation or pain, and is capable of sterilization and treatment by means of a built-in infrared sterilizer, thereby enabling natural urination and clean and hygienic maintenance.

### BACKGROUND ART

Generally, elderly patients, trauma patients, wheelchair patients, surgical patients, and patients with urinary incontinence cannot urinate on their own.

In particular, patients classified as seriously ill patients in hospitals are unable to do anything on their own, and therefore always need the help of others.

Among them, patients who can use their hands are not classified as seriously ill patients, but if the behavior is not possible, they can hardly do on their own.

For these patients, handling urine and feces is important in caring for the patients as much as eating.

Diapers are the most commonly used for this purpose.

Diapers cause harmful effects such as rashes and the like when worn for a long period of time, and patients cannot know exactly the time point when replacing diapers, which can lead to uremic toxin and make it more difficult for the patients.

In addition, since diapers are for one-time use, there is an uneconomical disadvantage in that several diapers must be used even a day for urinary treatment that is performed several times a day.

Thus, hospitals have recently recommended using a separate body urinal to ensure that urine is disposed of in a sanitary manner.

As the most commonly used body urinals for urine disposal, Korean Registered Utility Model No. 0177867 entitled “Body urinal for patients” introduces that a female-specific urinary collection container is provided at the upper part of the body urinal for male patients, the coupling groove of the urinary collecting container is connected to the inflow pipe of the body urinal for male patients, and the inlet of the urinary collecting tube is formed in the shape of a fallopian tube, so that the woman can sit on the inlet and urinate.

However, the above-mentioned conventional technology not only has the problem of leakage during urination because the inlet port does not fit properly against the woman's body, but also has the limitation that it can be used only when the patient expresses the intention to urinate.

Further, Korean registered U.S. Pat. No. 1,208,691 entitled “Body Urinal for Patients introduces a body urinal that adheres closely to the woman's body and forms a contact area that prevents urine leakage, but only when the patient expresses a desire to urinate, the patient or another person holds the handle and holds it close to the woman's body, which has the problem that it is impossible to use it for patients who cannot express the intention to urinate

Therefore, the present applicant has recently proposed the Korean Registered Patent No. 10-2300942 entitled “Free body-worn urinal for female patients,” which was previously filed and registered, but there are still deficiencies in patient comfort and hygiene. Therefore, there is a need to improve a body-worn urinal for female patients that is not only stable and comfortable for the patient to wear, but also hygienic.

### DETAILED DESCRIPTION OF THE INVENTION

### Technical Problem

Therefore, the present invention was designed to solve the conventional problems as described above, and an object of the present invention is to provide a body-worn urinal for female patients that attaches the body urinal so as to be tightly fixed to the genitals of the body, thereby achieving stable fixation and wearing in a sanitary diaper using a fixing band.

Another object of the present invention is to provide a body-worn urinal for female patients that not only provides convenience of use by using a Velcro band on the patient's pants, which is removable with Velcro, so that anyone can easily put on and take off the patient's pants, but also allows safe use by eliminating foreign body sensations and pressure even when the patient is lying down.

Another object of the present invention is to provide a body-worn urinal for female patients that provides a diagnostic test kit to a urine collection container that stores urine to check for hematuria, proteinuria, diabetes, etc. and confirm the health condition.

Yet another object of the present invention is to provide a body-worn urinal for female patients that can be immediately replaced in the shape of a urine collection container or a urine bag depending on the patient's condition that are lying down or moving in the structure of one body genital fixing device.

### Technical Solution

In order to achieve the above object, according to the present invention, there is provided a body-worn urinal for female patients comprising: a urination cup for tightly guiding female urination; a connecting adapter installed to be fastened to the front of the urination cup; a urine collecting tube, one end of which is fastened to the connecting adapter, and the other end of which has a spherical inner space, the urine collecting tube being installed such that urine is discharged through a drainage part inclined downwards in the space; a urine discharge tube connected/installed to discharge urine collected in the urine collecting tube; and a urine container connected to the urine discharge tube to temporarily store urine, wherein the body-worn urinal for a female patient further comprises: a far-infrared sterilizer mounted through one end of the upper side of the urine collecting tube and installed for sterilization and treatment; and a diagnostic test kit inserted/coupled to one side of the urine container for diagnostic test of urine.

Further, the urination cup further comprises a body that is oval in shape and is open at the rear so as to cover the female labia minora; a guide part that protrudes from one end of the body and is inserted into the female vagina to adjust the angle and position; a grid-type male and female fastening part formed in a screw shape on one inner peripheral surface of the body and screwed with the connection adapter; a Velcro attachment bands connected to both sides of one end of the body and coupled to a Velcro mounting portion; and a protruding ring part that is protrusively formed on both sides of the outer periphery of the body to connect and fix the Velcro attachment band.

Further, one end of the guide part may be formed to extend to a certain length, and is inserted into a female vagina; and the other end of the guide part may be configured with a diaphragm to prevent urine from flowing into the vagina and leaking from the side during urination.

The connection adapter may comprise an oval-shaped body with completely open front and rear sides and a step on the outside, a plurality of grid protrusion fastening parts with steps on the outside of the main body, wherein the urination cup and the urine collection tube are configured to be stored and fixed in the grid protrusion fastening part.

The urine collection tube may comprise an upper water collection part having a spherical inner space; a lamp holder formed through an upper end of one side of the water collection part; a threaded connection part formed to be open on one side so that the connection adapter is connected thereto; a drainage part formed downward from the water collection part to discharge urine; and a nipple that is formed at the end of the drainage part for inserting a urine discharge tube.

Further, the far-infrared sterilizer may be coupled with a lamp holder formed on one upper side of the urine collection tube, and the lamp holder may be configured to include an infrared lamp connected to a socket and a cable.

Further, the diagnostic test kit may comprise a urine test bottle that is inserted and mounted on one side of the upper part of the body of the urine container; and a urine test stick that is inserted into the inside of a urine test bottle and installed to apply urine.

Further, the urine test bottle may be further formed with a through hole that passes through both sides of the upper part so that one end of the urine discharge tube can be inserted and connected thereto.

The Velcro attachment band of the urination cup may be attached and fixed to the Velcro attachment portion of the patient diaper pants worn by the patient.

Further, the body-worn urinal for female patients may comprise a bag fixing holder which is configured to have a connecting locking jaw formed to protrude outward with a certain width at the lower end portion of the drainage part of the urine collection tube, and which forms a urine container removably attached to the drainage part of the urine collection tube, and is coupled and fixed to the connection locking jaw; a bag fixing cap on which the bag is inserted into the fixing holder on the upper side and which is fastened with screws; a urine plastic bag formed on the lower side to temporarily store urine; a bag guiding bag that puts into a urine plastic bag and fixes it to the leg during walking or rehabilitation exercise; a plurality of leg fixing bands that is connected to the upper, middle and lower ends of the bag guiding bag and fixed with Velcro; and a body urinal position fixing band that is connected to the uppermost end of the bag guiding bag, and attached and fixed to the Velcro attachment portion of the patient diaper pants.

### Advantageous Effects

The present invention can provide a body-worn urinal for female patients which attaches the body-worn urinal to be tightly fixed to the genitals of the body, thereby achieving stable fixation and wearing in a sanitary diaper using a fixation band.

Also, the present invention can provide a body-worn urinal for female patients that not only provides convenience of use by using a Velcro band on the patient's pants, which is removable with Velcro, so that anyone can easily put on and take off the patient's pants, but also allows safe use by eliminating foreign body sensations and pressure even when the patient is lying down.

In addition, the present invention has the effect of providing a diagnostic test kit to a urine collection container that stores urine, and checking for hematuria, proteinuria, diabetes, and the like to confirm the health condition.

Moreover, the present invention can provide a body-worn urinal for female patients that can be easily replaced in the form of a urine collection container or a urine bag depending on the state of the patient where the patient is lying down or moving in the structure of a body genital fixing device.

Further, the present invention has the effect of enabling disinfection and treatment by installing a far-infrared lamp at a location where the genitals of the body are fixed and urine is collected.

In other words, trauma patients who are being treated for dementia, paralysis, cancer, and the like while lying down is stripped of their diapers, put on AeroCool diaper pants, and then wear a far-infrared body urinal to collect and process urine while lying down, and then use the built-in low-temperature device to process it, and disinfect and treat with the built-in low-temperature infrared sterilizer for 10 to 20 minutes. In addition, it is a type of medical device that can check the patient's health condition by checking hematuria, proteinuria, diabetes, and the like using a diagnostic stick built into the urinal container, and infrared disinfection and treatment can be performed 1˜2 times/day depending on the patient's condition.

The present invention can provide an environmentally friendly medical device that eliminates a current difficult task that a patient's guardian comes face-to-face with the patient and replaces diapers 4-5 times a day, reduces discomfort, odor, leakage, and waste caused by diapers, allows low-temperature disinfection treatment to be performed once or twice a day, and improves urinary tract infections, inflammation, bedsores, and pain.

### BRIEF DESCRIPTION OF THE DRAWINGS

FIGS. 1 to 6 are embodiments showing a body-worn urinal for female patients according to the present invention, in which:

FIG. 1 is a diagram illustrating the use of a body-worn urinal for female patients according to the present invention in a mounted state;

FIG. 2 is an enlarged illustrative view of the main part of FIG. 1;

FIG. 3 is a diagram illustrating the use of a body-worn urinal for female patients according to the present invention in a worn state;

FIG. 4 is an enlarged illustrative view of the main part of FIG. 3;

FIG. 5 is an exemplary diagram showing a state in which the body-worn urinal for female patients according to the present invention is worn; and

FIG. 6 is an exemplary view showing an adult diaper and its wearing used in combination with a body-worn urinal for female patients according to the present invention.

### DETAILED DESCRIPTION OF THE EMBODIMENTS

While the present invention may be modified in various ways and take on various alternative forms, specific embodiments thereof are shown in the drawings and described in detail below. However, it should be understood that there is no intent to limit the present invention to the particular forms disclosed, but on the contrary, the present invention covers all modifications, equivalents, and alternatives falling within the spirit and scope of the present invention.

The present invention is not limited to the embodiments disclosed below, but may be implemented in various forms different from each other, and the present embodiment is provided so that this disclosure will be thorough and complete, and will fully convey the scope of the invention to those skilled in the art.

Hereinafter, preferred embodiments of the present invention will be described in detail with reference to the accompanying drawings. Throughout this specification, reference should be made to the drawings, in which the same reference numerals and symbols will be used to designate the same or like components. In the following description of the present invention, detailed descriptions of known functions and components incorporated herein will be omitted in the case that the subject matter of the present disclosure may be rendered unclear thereby.

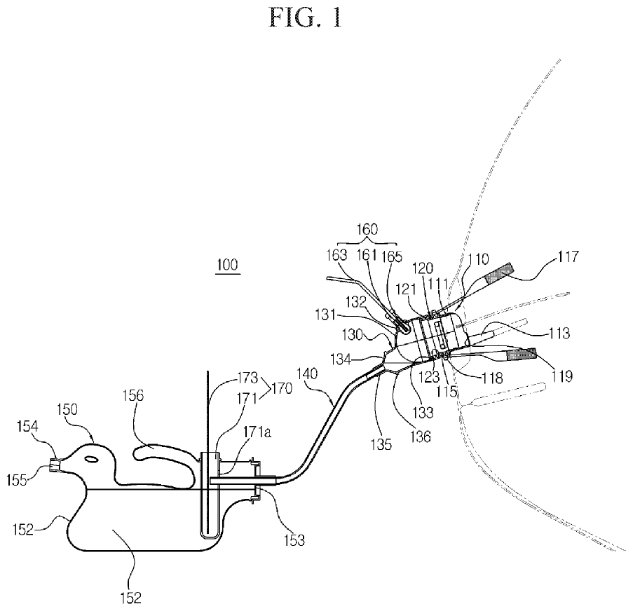

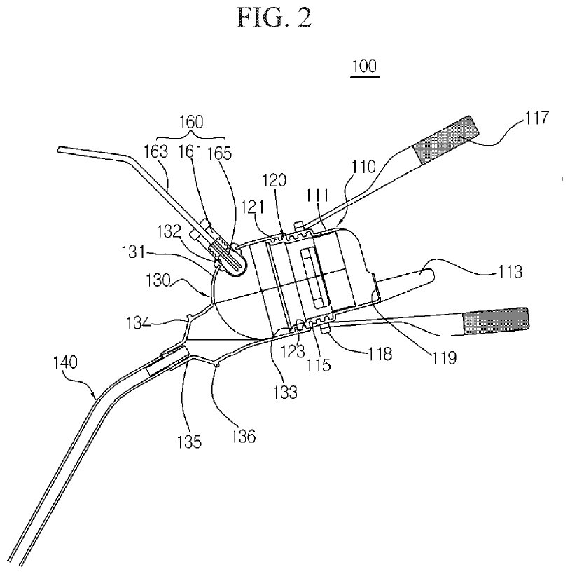

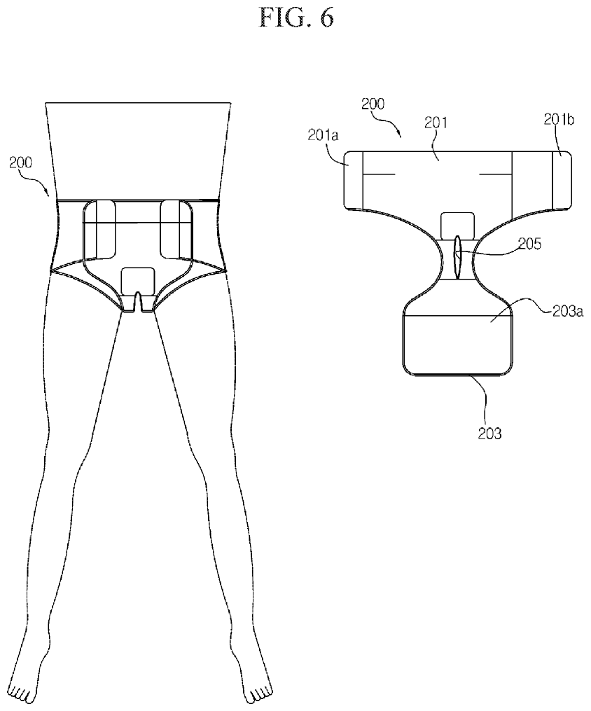

FIGS. 1 to 6 are embodiments showing a body-worn urinal for female patients according to the present invention.

Referring to FIGS. 1 to 5, the body urinal 100 for female patients according to the present invention comprises a urination cup 110 for tightly guiding female urination; a connecting adapter 120 installed to be fastened to the front of the urination cup 110; a urine collecting tube 130, one end of which is fastened to the connecting adapter 120, and the other end of which has a spherical inner space, the urine collecting tube being installed such that urine is discharged through a drainage part inclined downwards in the space; a urine discharge tube 140 connected/installed to discharge urine collected in the urine collecting tube; and a urine container 150 connected to the urine discharge tube 140 to temporarily store urine, and further comprises a far-infrared sterilizer 160 mounted through one end of the upper side of the urine collecting tube and installed for sterilization and treatment; and a diagnostic test kit 170 inserted/coupled to one side of the urine container for diagnostic test of urine.

The urination cup 110 may further comprise a body 111 that is oval in shape and is open at the rear so as to cover the female labia minora; a guide part 113 that protrudes from one end of the body 111 and is inserted into the female vagina to adjust the angle and position; a grid-type male and female fastening part 115 formed in a screw shape on one inner peripheral surface of the body 11 and screwed with the connection adapter 120; a Velcro attachment bands 117 connected to both sides of one end of the body 111 and coupled to a Velcro mounting portion; and a protruding ring part 118 that is protrusively formed on both sides of the outer periphery of the body 111 to connect and fix the Velcro attachment band 117.

Preferably, the urination cup 110 is made of silicone and has a soft oval shape, wherein the periphery of the end of the entrance toward the female genitalia is U-shaped, and a soft guide part 113 protrudes from the end, and can be composed of a diaphragm 119.

Here, the U-shaped end of the urination cup 110 is thinly cut outward in a fin-like manner so as to come into close contact with the contact skin surface, thereby preventing water leakage and pain. In addition, the urination cup 110 and the guide part 113 are simultaneously inserted into the female vagina and the labia minora, and the guide bar adjusts the angle and position of the labia minora urination cup, and when urinating, water is collected into the closely attached urination cup, and immediately passes through the drainage tube, reducing leakage. Further, an air hole is located at the upper part of the urination cup to prevent vacuum and allow for immediate drainage. Further, the diaphragm of the urinary cup has the effect of preventing urine from flowing into the vagina and preventing leakage.

The connection adapter 120 is provided to be fastened to the front of the urination cup 110, and is provided so as to connect the urine collection tube 130, and includes an oval-shaped body 121, and is formed of a stepped oval-shaped body 121 and a plurality of grid protrusion fastening parts 123 being stepping on the outside of the body 121, and the front and rear thereof may be configured to be completely open.

Here, the urination cup 110 is screwed to the grid protrusion fastening part 123 on one side being stepped, and the urine collection tube 130 may be coupled to the grid protrusion fastening part 123 on the other side being stepped by screw fastening.

Meanwhile, the connection adapter 120 is oval-shaped, wherein the outer side is formed in the form of male and female grid type protrusion, one side of which is fitted inside the urination cup 110, and the other side of which is fitted inside the urine collection tube 130. However, the urination cup and the urine collection tube that are fitted on the outside are made of elastic rubber, and can be assembled by pulling and stretching the inside when they are fitted. In addition, the thin oval-shaped connection adapter that fits inside the urination cup prevents the soft urination cup from narrowing when the urinal is inserted into the labia minora. It is preferable that the inner skeleton be made of a material with some elasticity so that it can be received without clogging when urine comes out of the urethra.

The urine collection tube 130 may comprises an upper water collection part 131 having a spherical inner space, a lamp holder 132 formed through an upper end of one side of the water collection part 131, a threaded connection part 133 formed to be open on one side so that the connection adapter 120 is connected thereto, a drainage part 134 formed downward from the water collection part 131 to discharge urine, and a nipple 135 that is formed at the end of the drainage part 134 for inserting a urine discharge tube 140.

Preferably, the water collection part 131 has a spherical shape whose interior is empty, wherein the empty interior can be formed sot that urine jetted from the genitals can be collected. One side of the spherical water collection part 131 is formed with a threaded connection part 133 by opening with a certain diameter and length on the outside, wherein such a water collection part 131 is opened downward with a certain diameter and length, thereby forming a drainage part 134 that discharges urine, and the lower part of the drainage part 134 may be formed with a nipple 125 that extends and protrudes downwardly to a certain length with a smaller diameter.

In addition, the lower end of the drainage part 134 is further formed with a connection hooking jaw 136 which is protrusively formed outward to a certain width, so that the upper end of the urine container or urine bag directly connected to the drainage part 134 wraps around the outer peripheral surface of the drainage part 134, thereby fixing the upper part of the urine collection bag using a fixing means such as a Velcro band or rubber band.

In addition, the water collection part 131 further has a lamp holder 132 that is fitted into a portion formed through the daily side, wherein such a lamp holder 132 may be equipped with a far-infrared sterilizer 160 having an infrared lamp 165 connected to the socket 161 and a cable 163. As a result, it is possible to collect and process urine while lying down using a far-infrared sterilizer, and then disinfect and treat with the built-in low-temperature infrared sterilizer for 10 to 20 minutes.

Here, the lamp holder 132 can provide a structure which remove the lamp through a detachment/attachment method that allows the lamp to be mounted and then dismantled only during infrared sterilization, and which acts as a ventilation for air to pass through the bottom hole of the holder when disinfection is not performed. When the patient lies on the left or right side, the urine collection tube can be rotated left or right by the connection part. Even if the patient lies on the side too much and urine accumulates towards the lamp holder, water leakage occurs only when exceeding the height of the hole in the lamp holder, which makes it difficult for leakage to occur.

The urine discharge tube 140 is a moving passage that discharges urine into the urine container 150, and it is more desirable to form of a material that is easy to bend and deform and has good restoring force even when bent.

The urine container 150 includes a body 151, a urine storage space 152 that can store a certain amount of urine inside, a drain cap 153 that is fixed by fitting coupling so that the other end of the urine discharge tube 140 is connected on one side of the body 151, a discharge port 154 that allows urine filled inside to be discharged to the outside on the other side of the body 151, a drain air hole 155 mounted to open and close the discharge port 154, and a handle 156 that the user can hold with hand.

Here, the urine discharge tube is precisely inserted into the central pipe of the drain cap to prevent it from falling out, thereby stably leading to urination.

The diagnostic test kit 170 may comprise a urine test bottle 171 inserted through and mounted on one side of the upper end of the body 151 of the urine container 150, and a urine test stick 162 that is inserted into the inside of the urine test bottle 171 and is provided to apply urine. At this time, the urine test bottle 171 may be formed with through holes 171a on both sides of the upper part, and allow urine to flow by inserting and connecting one end of the urine discharge tube 140.

On the other hand, it is more preferable that the urine container 150 has a size that can sufficiently store at least two doses of urine, and a urine storage space 152 is formed, and the bottom surface is formed with a flat surface of a sufficient size to enable stable landing anywhere. For this purpose, the urinal container 150 may be formed in the shape of various more familiar animals or livestock, including a duck shape as shown in the illustration, but is not limited thereto.

Therefore, the urine container 150 can be formed close to the patient or at a farther distance depending on the length of the urine discharge tube 140.

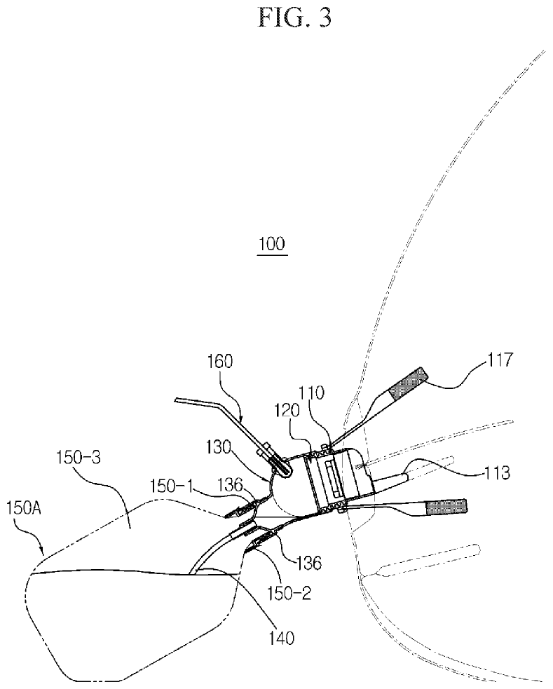

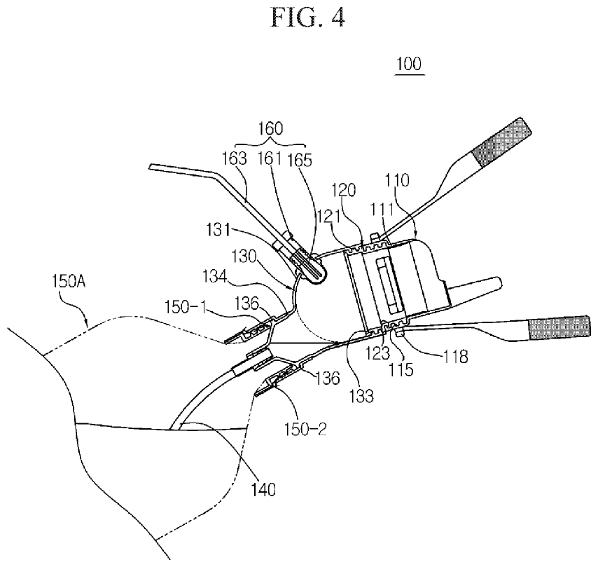

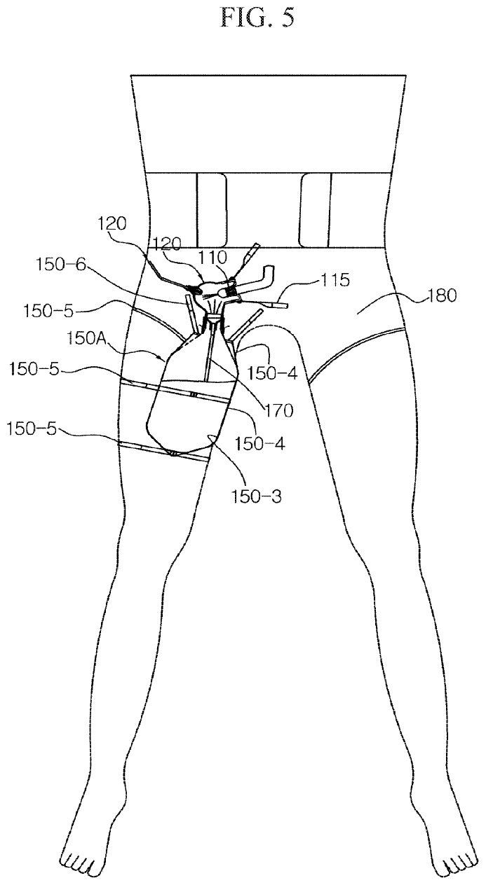

FIG. 3 is a diagram illustrating the use of the body-worn urinal for female patients according to the present invention in a worn state; FIG. 4 is an enlarged illustrative view of the main part of FIG. 3, and FIG. 5 is an exemplary diagram showing a state in which the body-worn urinal for female patients according to the present invention is worn.

Referring to FIGS. 3 to 5, the body-worn urinal for female patient may comprise a bag fixing holder 150-1 which is configured to have a connecting locking jaw 136 formed to protrude outward with a certain width at the lower end portion of the drainage part 124 of the urine collection tube 120, and which forms a urine container 150A, and is coupled and fixed corresponding to the connection locking jaw 136; a bag fixing cap on which the bag is inserted into the fixing holder 150-1 on the upper side and which is fastened with screws; a urine plastic bag 150-3 formed on the lower side to temporarily store urine; a bag guiding bag 150-4 that puts into a urine plastic bag and fixes it to the leg during walking or rehabilitation exercise; a plurality of leg fixing bands 150-5 that is connected to the upper, middle and lower ends of the bag guiding bag 150-4 and fixed with Velcro; and a body urinal position fixing band 150-6 that is connected to the uppermost end of the bag guiding bag 150-4, and attached and fixed to the Velcro attachment portion of the patient diaper pants.

Here, the bag guiding bag 150-4 is a bag that puts a plastic urine bag in the bag guiding bag and fixes it to the leg when walking, during rehabilitation, or while moving in a wheelchair. The urinal position fixing band at the top of the entrance is fixed by attaching it to the diaper panties, and the lower and middle parts use the elastic Velcro band of the leg fixing band to fasten and secure the band to the thigh above the knee, so urination can be performed without sloshing, leakage, and withdrawal while allowing the movement freely.

On the other hand, the urine collection tube 130 is formed with a nipple 135 at the lower part of the drainage part 124 with a smaller diameter and protrudes downwardly to a certain length, and the nipple 135 can be connected to a straw-shaped urine drain tube 140.

On the other hand, the Velcro attachment band 117 of the urination cup 110 may be formed to be attached and fixed to the Velcro attachment portion 201 of the patient diaper panties 200 worn by the patient.

Referring to FIG. 5, the patient diaper pants 200 may comprise a back plate band 201 formed to protrude on both sides and wrap around the back plate having both left and right wings 201a and 201b having a Velcro structure; and a front plate shielding band 203 that is formed to have a constant width in the front while being connected and formed to a back plate band 201 with one connecting band, thereby covering the patient's genitals, wherein the front plate shielding band 203 is configured such that its front surface portion is composed of a Velcro attachment portion 203a, and its center is formed with a hole 205 that connects the patient's genitalia.

Here, a body-worn urinal 100 for female patients is installed around the hole 205 from which the body's genitals are opened, and the Velcro attachment band 117 of the urination cup 110 can be tightly fixed to the Velcro attachment portion 203a of the front plate shielding band 203.

The functions and effects of the body-worn urinal for female patients of the present invention will be described as follows. The following description is based on the above structural description.

First, in the body-worn urinal 100 for female patients of the present invention, the urination cup 110 is inserted into the labia minora area of the female body and worn.

Next, the urination cup 110 is detached and installed on the urine collection tube 120.

Then, while wearing the patient diaper pants 200, the patient can finish wearing the diaper by grasping both ends of the Velcro attachment band 117 that is connected and fixed to the urination cup 110 so that it is attached and fixed to the Velcro attachment portion 203a of the patient diaper pants 200.

Even when the patient is in various pasture such as lying down during such a wearing condition, it is possible to stably perform urination through the urethra while maintaining a stable fixation state.

The urine released through the patient's urethra is sprayed into the water collection part 131 of the urine water collection tube 130 formed in a spherical shape in front of the urethra, and is discharged through the urine discharge tube 40 which is directly connected to the nipple 135 via the drainage part 134 along the main surface.

Here, urine is urinated into the urination cup, and the urine collection tube stores the initial amount of urine for a while, and then when the urine becomes weak, the entire amount can be discharged. The capacity of the urine collection tube is preferably large enough to collect the amount of initial urine inflow.

Urine discharged from the urine collection tube 130 through the urine discharge tube 140 is recovered in the urine container 150, and at least 2 doses of urine are collected in the urine container 150.

Once the urine is collected, the end of the urine discharge tube 140 is separated from the urine container 150, and the drain stopper 155 that blocks the discharge port 154 is separated to allow urine to be discharged in a toilet or the like.

The urinal container 150 that discharges urine filled inside can be reused by washing with water.

On the other hand, the far-infrared sterilizer 160 mounted on the urine collection tube 130 can disinfect and sterilize the area around the patient's urethra once or twice a day (for 10 to 20 minutes) depending on the condition.

In addition, the diagnostic test kit 170 built into the urine container can check the health status by checking for blood in blood urine, proteinuria, diabetes, and the like with a urine test stick. Alternatively, during detailed examinations, it will be possible to provide an environmentally friendly medical device that can remove the built-in test bottle, transfer it, and send it to a hospital.

Further, when used by intensive care patients or general patients at night, a body-worn urinal for female patients that uses a urine discharge tube in a stationary manner connects the urine discharge tube to the drainage nipple and connects it to the plastic urine tank on the lower side to urinate. As a result, the guide part and the urination cup are inserted into the vagina and the labia minora at the same time, and the guide adjusts the angle and position of the labia minora urination cup, so that when urinating, water is collected through the closely attached urination cup, and is immediately discharged into the discharge tube, thereby reducing leakage. There is an air hole at the top of the urination cup to prevent vacuum and allow immediate drainage. The diaphragm of the urination cup is effective in preventing urine from entering the vagina and preventing leakage. The water collection tube acts as a service tank to collect and transfer urine and prevents backflow of urine. When draining, the patient can know the state of urination by making a “pop” sound from the air hole.

On the other hand, body-worn urinals for general patients, wheelchair patients, or mobile female patients used during rehabilitation exercise or walking is used by providing a urine container in the form of a plastic bag, and can be used stably by wearing it with Velcro on the thighs of a patient wearing diaper panties.

Currently, general patients are unable to lie down, but move to left or right or move their legs a lot. Alternatively, the patients use ropes while doing rehabilitation exercises or walking because they are cumbersome and twisted, so they use plastic urine bags. In addition, not only is it convenient to use a plastic bag when moving a patient in a wheelchair, but also the urine is collected by inserting the plastic bag into the bag cap and rotating the cap to the fixed holder. When sleeping at night when there is little activity, the patient can urinate by changing to a urinal container that can collect more urine. While the present invention has been shown and described in connection with the exemplary embodiments, it will be apparent to those skilled in the art that modifications and variations can be made without departing from the spirit and scope of the invention as defined by the appended claims.

1. A body-worn urinal for female patients comprising:
a urination cup for tightly guiding female urination;
a connecting adapter installed to be fastened to the front of the urination cup;
a urine collecting tube, one end of which is fastened to the connecting adapter, and the other end of which has a spherical inner space, the urine collecting tube being installed such that urine is discharged through a drainage part inclined downwards in the space;
a urine discharge tube connected/installed to discharge urine collected in the urine collecting tube; and
a urine container connected to the urine discharge tube to temporarily store urine,
wherein the body-worn urinal for a female patient further comprises: a far-infrared sterilizer mounted through one end of the upper side of the urine collecting tube and installed for sterilization and treatment; and a diagnostic test kit inserted/coupled to one side of the urine container for diagnostic test of urine.

2. The body-worn urinal for female patients as claimed in claim 1, wherein the urination cup further comprises,
a body that is oval in shape and is open at the rear so as to cover the female labia minora;
a guide part that protrudes from one end of the body and is inserted into the female vagina to adjust the angle and position;
a grid-type male and female fastening part formed in a screw shape on one inner peripheral surface of the body and screwed with the connection adapter;
a Velcro attachment bands connected to both sides of one end of the body and coupled to a Velcro mounting portion; and
a protruding ring part that is protrusively formed on both sides of the outer periphery of the body to connect and fix the Velcro attachment band.

3. The body-worn urinal for female patients as claimed in claim 2, wherein:
one end of the guide part is formed to extend to a certain length, and is inserted into a female vagina; and
the other end of the guide part is configured with a diaphragm to prevent urine from flowing into the vagina and leaking from the side during urination.

4. The body-worn urinal for female patients as claimed in claim 1, wherein
the connection adapter comprises,
an oval-shaped body with completely open front and rear sides and a step on the outside,
a plurality of grid protrusion fastening parts with steps on the outside of the main body,
wherein the urination cup and the urine collection tube are configured to be stored and fixed in the grid protrusion fastening part.

5. The body-worn urinal for female patients as claimed in claim 1, wherein
the urine collection tube comprises,
an upper water collection part having a spherical inner space;
a lamp holder formed through an upper end of one side of the water collection part;
a threaded connection part formed to be open on one side so that the connection adapter is connected thereto;
a drainage part formed downward from the water collection part to discharge urine; and
a nipple that is formed at the end of the drainage part for inserting a urine discharge tube.

6. The body-worn urinal for female patients as claimed in claim 1, wherein
the far-infrared sterilizer is coupled with a lamp holder formed on one upper side of the urine collection tube, and
the lamp holder is configured to include an infrared lamp connected to a socket and a cable.

7. The body-worn urinal for female patients as claimed in claim 1, wherein
the diagnostic test kit comprises,
a urine test bottle that is inserted and mounted on one side of the upper part of the body of the urine container; and
a urine test stick that is inserted into the inside of a urine test bottle and installed to apply urine.

8. The body-worn urinal for female patients as claimed in claim 1, wherein:
the urine test bottle is further formed with a through hole that passes through both sides of the upper part so that one end of the urine discharge tube can be inserted and connected thereto.

9. The body-worn urinal for female patients as claimed in claim 1, wherein:
the Velcro attachment band of the urination cup is attached and fixed to the Velcro attachment portion of the patient diaper pants worn by the patient.

10. The body-worn urinal for female patients as claimed in claim 1, comprising:
a bag fixing holder which is configured to have a connecting locking jaw formed to protrude outward with a certain width at the lower end portion of the drainage part of the urine collection tube, and which forms a urine container removably attached to the drainage part of the urine collection tube, and is coupled and fixed to the connection locking jaw;
a bag fixing cap on which the bag is inserted into the fixing holder on the upper side and which is fastened with screws;
a urine plastic bag formed on the lower side to temporarily store urine;
a bag guiding bag that puts into a urine plastic bag and fixes it to the leg during walking or rehabilitation exercise;
a plurality of leg fixing bands that is connected to the upper, middle and lower ends of the bag guiding bag and fixed with Velcro; and
a body urinal position fixing band that is connected to the uppermost end of the bag guiding bag, and attached and fixed to the Velcro attachment portion of the patient diaper pants.
